(19) United States
(12) Patent Application Publication
(10) Pub. No.: US 2005/0020963 A1
(43) Pub. Date: Jan. 27, 2005

(54) Implantable duct system connecting the intrahepatic portal vein to the femoral vein for establishing a subcutaneous porto-systemic shunt and simultaneously providing a durable access to the portal vein

(76) Inventor: Abdelwahab M. Gabal, (US)

Correspondence Address: EUGENE LIEBERSTEIN;ANDERSON KILL + OLICK, P.C., 1251 AVENUE OF THE AMERICAS, NEW YORK, NY, 10020, US

(21) Appl. No.: 10/869,107

(22) Filed: Jun. 15, 2004

(30) Foreign Application Priority Data
Jul. 26, 2003 (EG) 2003070721

(51) Int. Cl. A61M 5/00; A61M 31/00

(52) U.S. Cl. 604/8; 623/23.64

(57) ABSTRACT

A set (FIG. 1&2) designed to provide a durable access to the portal vein and to divert portal blood to the systemic circulation in order to relieve congested portal system. The set is composed of a duct (1) and a shunt tube (7). The duct is composed of a covered flexible tubular braid with two opened ends. One end has flaring edges and forms the inner opening (2) while the other end is sealed to form a hub (3). To insert the duct into the intrahepatic portal vein through the percutaneous route the duct is mounted over a puncture needle (13) and contained in a small-constrained diameter by means of a peel-away sheath (16). After the position of the inner opening of the duct has been adjusted to the desired location the said sheath is peeled away to allow the duct to expand. The shunt tube (7) is composed of a long flexible vascular graft (8) equipped at its upper end with a head (9) while its lower end is free to be sutured with the femoral vein (21). The said head is cupped at the inner side (10) to fit in the hub (3) of the said duct (1), while back of the said head has a window covered with an elastic membrane (11). This shunt tube is applied in case both a portal access and a porto-systemic shunt are required. In case of only a durable portal access is needed, a plug (4) with a central window covered by an elastic membrane (6) is applied to the hub of the duct. This prevents bleeding from the duct and simultaneously allows entrance of needles, catheters, etc. to the duct and portal vein.

### BACKGROUND OF THE INVENTION

Portal vein transfers portal blood from intestine and spleen to the liver where several chemical and biological processes take place before this blood passes into the general venous circulation through the hepatic veins.

Diseases that affect liver parenchyma like liver cirrhosis and periportal fibrosis impede flow of portal blood through the liver. This leads to portal hypertension. Bleeding esophageal varices, ascites and encephalopathy are the end results of portal hypertension.

For treatment of portal hypertension several surgical interventions have tried to decompress the portal system by establishing a shunt between portal vein or one of its branches and systemic venous circulation. The basic idea is to establish a communication or create a channel (shunt) between the portal and the systemic venous circulation, thus allowing the blood to be shunted from the high pressure within the portal venous system to the low pressure in the systemic venous circulation.

The widely accepted method for non-surgical creation of such a shunt is called Transjugular Intrahepatic Portosystemic Shunt (TIPS). In this procedure a tunnel is created within the liver parenchyma connecting the high pressure-portal vein (portal venous circulation) and the low pressure-hepatic vein (systemic venous circulation). Subsequently, blood from the portal vein is allowed to flow into the hepatic vein thereby reducing the portal venous pressure and relieve the portal congestion. Walls of this tunnel are strengthened by a synthetic prosthesis called stent (graft), to prevent its collapse. This procedure has the following advantages:
1. It can be performed in sick patients who can not tolerate major surgery.
2. There is no need for general anesthesia.
3. It can be performed as an emergency procedure in patients with active variceal bleeding.
4. It is less invasive and less costly as compared to surgical shunts.

However, prolonged follow-up studies have revealed several complications and limitations. These include the following:
1. The procedure is technically difficult and needs considerable experience to perform.
2. The path of the puncture set from the internal jugular vein to the hepatic vein traverses the right atrium of the heart, which increases risk of the procedure.
3. Frequent thrombosis of the stent resulting in occlusion of the shunt tract.
4. Difficult to be applied in patients with fibrotic livers, like schistosomal liver disease, as the puncture set used to open the tunnel is unable to penetrate the fibrotic liver tissue.
5. The hepatic vein is used as the draining vein connecting the shunt to the inferior vena cava. This hepatic vein is relatively small in caliber and its thin walls cannot adapt well to the high pressure of the portal blood flow. These factors frequently lead to occlusion of this vein and subsequently the shunt tract.
6. Re-canalization of the occluded shunt tract is not feasible in most cases.

### BRIEF SUMMARY

A duct system designed for gating a durable easy access to the portal vein (exteriorization of the portal vein) and treatment of portal hypertension. The system connects intrahepatic portion of the portal vein to the femoral vein allowing shift of blood from the congested portal vein into the systemic circulation.

The set is composed of a duct and a shunt tube. The duct (1) is composed of tubular braid constructed from highly flexible metal (Nitinol) and covered by a thin membrane of expanded Polytetrafluoroethylene (ePTFE). The said duct is opened at its both ends, one opening has flaring edges and forms the front or inner opening (2) while the other end is sealed to a hub (3). The said duct is mounted over a puncture needle (13) and its diameter is constrained by means of a peel-away sheath (16) to allow penetration of the liver and portal vein through the percutaneous route. After reaching the planned location puncture needle is withdrawn and the sheath is peeled away allowing the duct to expand.

The shunt tube (7) is formed of a vascular graft (8) made of expanded Polytetrafluoroethylene (ePTFE) and equipped at its upper end with a cupped head (9) fitting snugly over the said hub (3) of the said duct (1). The head is provided with a window (11) covered by elastic membrane. The lower end (12) of the said vascular graft is free to be sutured with the femoral vein (21). A removable plug (4) formed of synthetic material is provided to be applied to the hub (3) of the duct in case of a durable access to the portal vein is required without establishing porto-systemic shunt.

### DESCRIPTION OF THE DRAWINGS

FIG. 1

The duct is composed of a tubular braid (1) of Nitinol. Inner end (2) of this tubular braid has a flaring edges while the other end is sealed to a hub of synthetic material (3).

A removable plug formed of synthetic material (4) has a cupped mouth (5) that matches the size and diameter of the said hub (3). Back of the said plug is a window covered by a highly elastic membrane (6) through which catheters, guidewires, needles etc. can be introduced.

FIG. 2

Shunt tube (7) is composed of a flexible vascular graft (8) resist compression equipped with a hollow head (9) at one end while the other end (12) is free. The said head has a mouth (10) that matches the shape and diameter of the said hub (3). Back of the said head is a window covered by elastic membrane (11) through which catheters, guidewires, needles etc. can be introduced.

FIG. 3

Shows the mounted shunt set. Head (9) of the shunt tube (7) is applied to the hub of the duct (1) to form a continuous channel.

FIG. 4

Shows the duct when used as a durable access (port) to the portal vein. The plug is applied to the hub (3) of the duct (1). Back of the plug is opened and covered by a highly flexible elastic membrane to form a window (6) through which catheters, guidewires, needles etc. can be introduced.

FIG. 5

Puncture needle (13) composed of a metal cannula (14) and a needle (15) inserted within it. The duct (1) is mounted over the said cannula (14) and constrained to a small diameter by means of a sheath (16). The said sheath has a fissure (17) at its back and equipped with two side-arms (18) to provide a peel-away mechanism for removal of the said sheath (16) after percutaneous penetration of the intrahepatic portal vein.

FIG. 6

Diagram showing coronal section of the liver and abdominal wall with the shunt set in place. The duct (1) is located within the liver (19) with the inner end (2) of the duct opens into the portal vein (20). The shunt tube (7) is connected to the hub of the duct and located in the subcutaneous space (22) and covered by the skin (23).

FIG. 7

Diagram showing near coronal view of the duct (1) within the liver (19) with its inner opening (2) inside portal vein (20) while its hub (3) locates in the subcutaneous space (22). The plug (4) is applied to the said hub and covered by the skin (23). The plug prevents blood leakage and its window (6) provides access to the duct (1).

FIG. 8

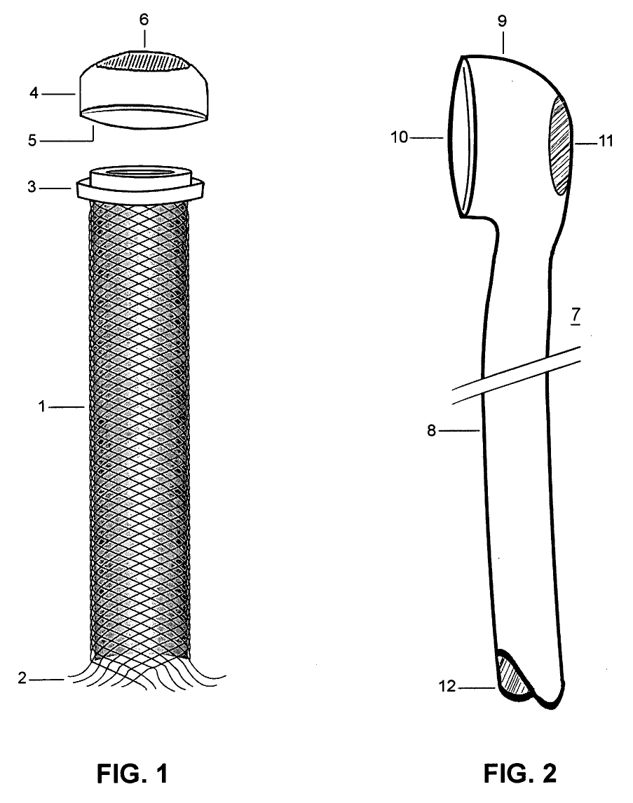

Final appearance of the shunt set after it has been properly positioned. The duct (1) diverts blood from the portal vein (20) into the right femoral vein (21) through the shunt tube (7).

### DETAILED DESCRIPTION OF THE INVENTION

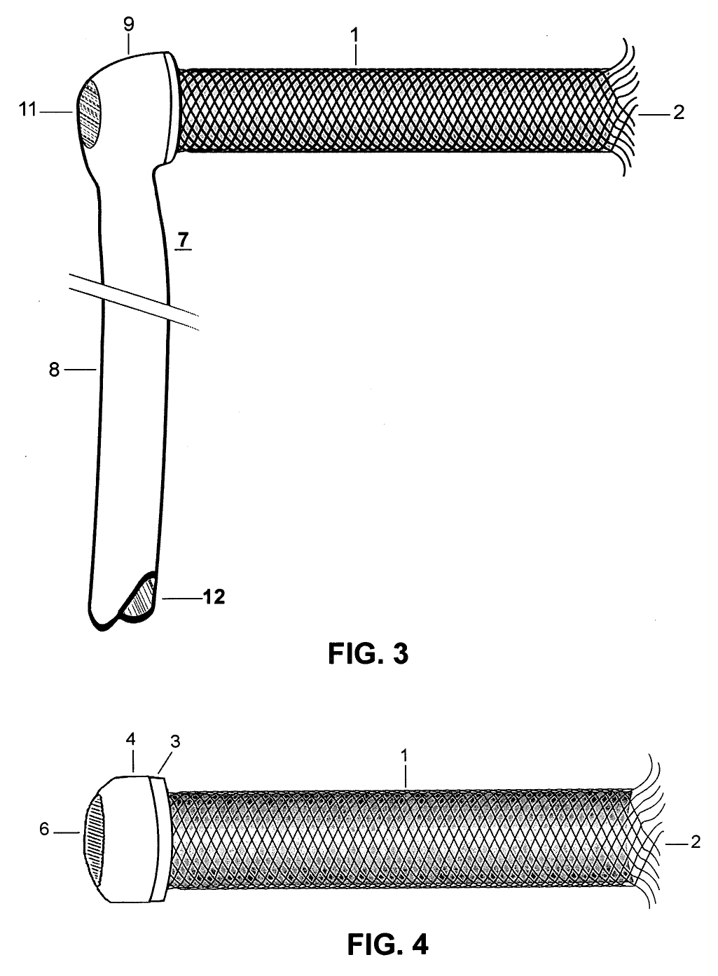

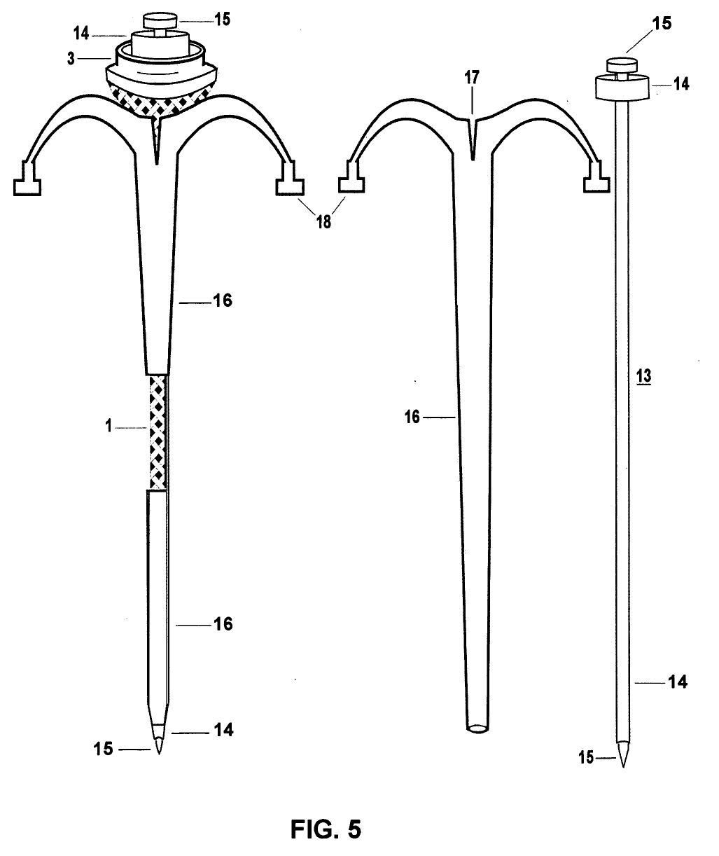

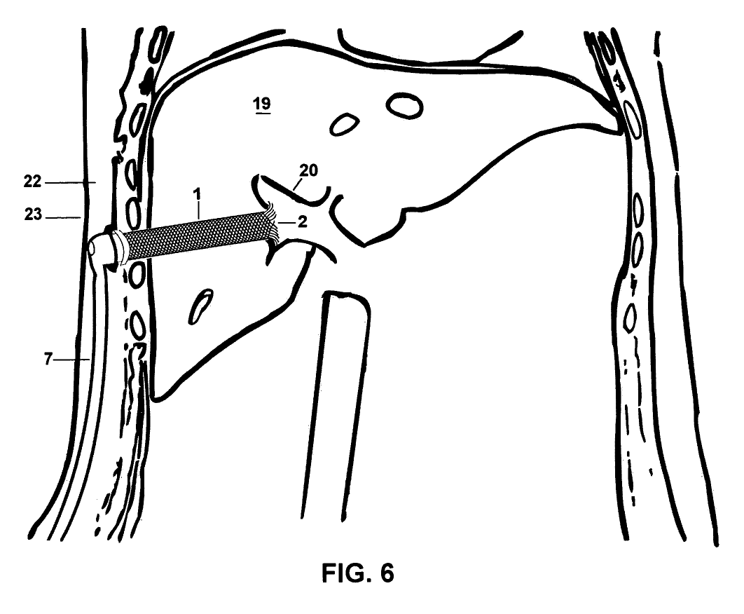

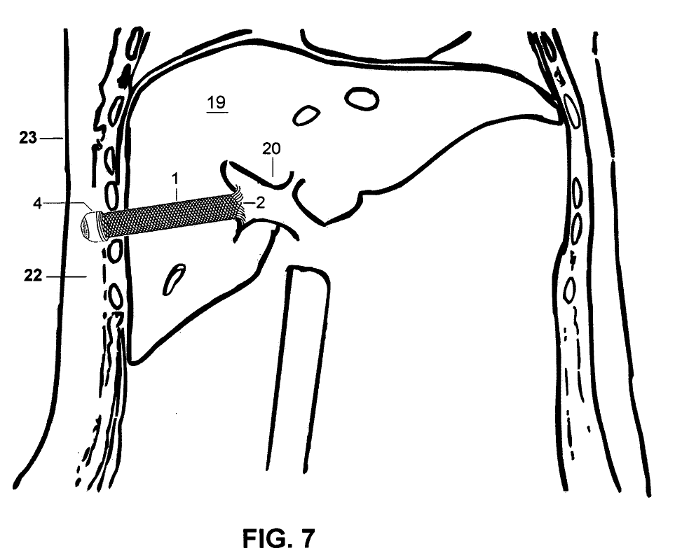

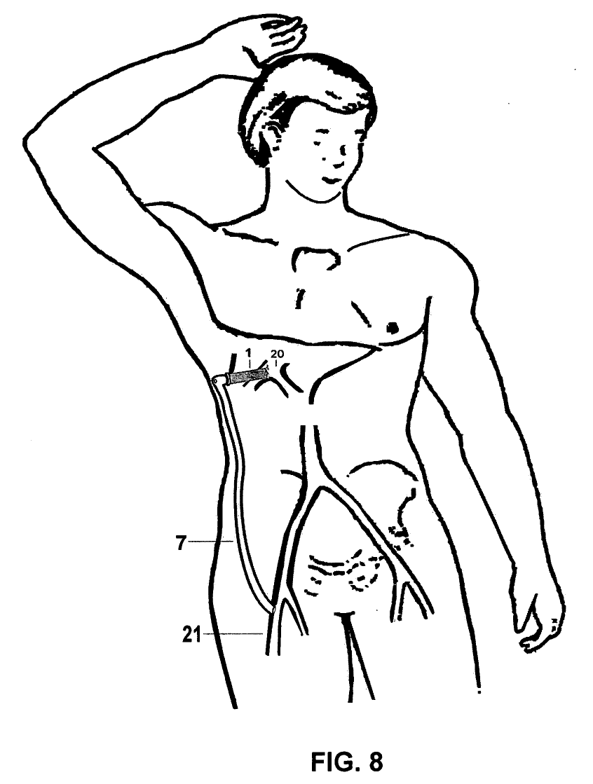

The system is composed of: a duct (1), and a shunt tube (7) when attached together they form continuous hollow channel (FIG. 3).

The said duct (1) is a tubular mesh constructed from highly flexible metal (Nitinol) and covered by a thin membrane of expanded Polytetrafluoroethylene (ePTFE). The said duct has two opened ends; the inner end (2) is formed by the edges of the said tubular mesh (1) which take a flaring form to provide internal fixation mechanism of the duct (1) within the portal vein (20). The outer end of the said duct is tightly soldered to a rounded hub (3). A cup-shaped plug (4) is provided to be applied to the hub (3) of the said duct (1) to prevent bleeding from the duct when it used as a durable port to the portal vein. The said plug (4) has a concave opened end (5) which matches the shape and diameter of the said hub (3). Back of the said plug has a window covered by a highly elastic membrane (6). It is designed to serve as a permeable entrance allowing passage of needles, catheters, guidewires, etc. into the said duct and portal vein without loss of blood. In case of porto-systemic shunt is planned, the said plug is not used.

The said shunt tube (7) is composed of a vascular graft (8) equipped at its upper end with a hollow head (9) while the lower end (12) is free and can be sutured to the femoral vein (21). The said head has a cup-shaped mouth (10) which matches with the said hub (3) of the said duct (1). Shape and diameter of the said mouth closely fits into the hub (3). An opening (11) is provided on the back of the head. A highly flexible membrane covers this opening. Needles, catheters, guidewires, etc. can be introduced via this window into the shunt system including portal vein without blood loss.

In order to introduce the said duct (1) into the portal vein (20) through the percutaneous route, the said duct is mounted over a metal needle (13) and compressed into a constrained diameter by means of a peel-away sheath (16). The said puncture needle (13) is composed of a cannula (14) and a tapered-tip trocar (15).

Advantages of this Invention:

1. Presence of subcutaneous port provides durable direct access to the portal vein i.e. exteriorization of the portal vein allowing several procedures to be accomplished, like monitoring of pressure in the portal vein, liver dialysis, removal of thrombus in the portal vein, re-canalization of the tract in case of stenosis or occlusion, taking sample from portal blood for laboratory studies, etc.

2. Shunt tube connects intrahepatic portion of the portal vein to the femoral vein. This vein has a larger caliber and a stronger wall than the hepatic veins used in the conventional TIPS. Therefore femoral vein can adapt to the high pressure of the flowing portal blood much better than the hepatic veins.

3. Shunt tube can also be connected to the subclavian vein providing more option for shunt draining veins.

4. Technically simpler than the conventional TIPS procedure. For an interventional radiologist very little additional training is required to access the intrahepatic portal vein from the percutaneous route.

1) An implantable set of ducts used to give a durable port to the portal vein and to establish subcutaneous porto-systemic shunt composed of duct and shunt tube.

2) An implantable durable shunt set composed of a duct equipped with a plug. The said duct is expandable tubular mesh constructed from highly flexible metal and covered by a thin membrane of synthetic material. The said duct has one opening formed by the flaring edges of the filaments of the said mesh and a hub of synthetic material on the other end. The said duct is compressed around a puncture needle into a small diameter by means of a sheath in order to introduce the said duct into the liver. The said sheath is designed in the way to allow its removal using peel-away technique. The said duct is equipped with a removable plug which matches the said hub of the said duct. The said plug has a window at its back covered by elastic membrane allowing introduction of catheters, needles, guidewires and recanalization tools into the duct and portal vein.

3) An implantable durable shunt set composed of duct and shunt tube. The shunt tube is constructed from a long, flexible and uncompressible pipe made of expanded polytetrafluoroethylene (ePTFE) and equipped with a hollow head at one end. The said head matches the shape and diameter of the hub of the said duct and fits in snugly to provide a watertight connection. On the back of the said head is a window covered by elastic membrane to give a port to the entire shunt set including the portal vein and its branches. Other end of the shunt tube is free and can be connected to a blood vessel.
